(19) United States
(12) Patent Application Publication
(10) Pub. No.: US 2008/0033338 A1
(43) Pub. Date: Feb. 7, 2008

(54) ELECTROOSMOTIC PUMP APPARATUS AND METHOD TO DELIVER ACTIVE AGENTS TO BIOLOGICAL INTERFACES

(76) Inventor: Gregory A. Smith, Sammamish, WA (US)

Correspondence Address: SEED INTELLECTUAL PROPERTY LAW GROUP PLLC, 701 FIFTH AVE, SUITE 5400, SEATTLE, WA, 98104, US

(21) Appl. No.: 11/616,666

(22) Filed: Dec. 27, 2006

Related U.S. Application Data
(60) Provisional application No. 60/754,943, filed on Dec. 28, 2005.

(51) Int. Cl. A61N 1/30

(52) U.S. Cl. 604/20

(57) ABSTRACT

A device uses electroosmotic assistance in the delivery of an active agent, such as drugs, to a biological interface. The active agent is delivered from a flexible impermeable reservoir by compressing the reservoir. The compression is caused by electroosmotically pumping fluid into a chamber that surrounds the reservoir, thereby increasing the pressure in the chamber. The increased pressure in the chamber causes the reservoir to compress and expel the active agent stored therein.

### CROSS REFERENCE TO RELATED APPLICATION

The present application claims priority from and the benefit under 35 U.S.C. § 119(e) of U.S. Application Ser. No. 60/754,943, entitled “ELECTROOSMOTIC PUMP APPARATUS AND METHOD TO DELIVER ACTIVE AGENTS TO BIOLOGICAL INTERFACES,” filed Dec. 28, 2005, assigned to the same assignee as the present application, and incorporated herein by reference in its entirety.

### TECHNICAL FIELD

This disclosure generally relates to devices that deliver active agents, and more particularly but not exclusively, relates to the delivery of active agents, such as therapeutic agents or drugs, with the assistance of electroosmosis.

### BACKGROUND INFORMATION

There are several types of devices that use electromotive force to deliver active agents to a patient. For example, an iontophoretic device employs an electromotive force to transfer an active agent such as an ionic drug or other therapeutic agent to a biological interface, for example skin or mucus membrane.

Iontophoresis devices typically include an active electrode assembly and a counter electrode assembly, each coupled to opposite poles or terminals of a power source, for example a chemical battery. Each electrode assembly typically includes a respective electrode element to apply an electromotive force. Such electrode elements often comprise a sacrificial element or compound, for example silver or silver chloride.

The active agent may be either cation or anion, and the power source can be configured to apply the appropriate polarity based on the polarity of the active agent. Iontophoresis may be advantageously used to enhance or control the delivery rate of the active agent. The active agent may be stored in a reservoir such as a cavity. Alternatively, the active agent may be stored in a reservoir such as a porous structure or a gel. An ion exchange membrane may be positioned to serve as a polarity selective barrier between the active agent reservoir and the biological interface.

There are other types of devices and products that may be used to transfer or otherwise exchange active agents with a biological interface. Common examples include various types of syringes, dialysis equipment, pumps, medicated patches (such as nicotine patches or birth control patches), and the like. In other situations, active agents may be delivered directly to the patient without necessarily requiring electrical, mechanical, or chemical assistance. For instance, the patient may ingest encapsulated pills having an outer coating that is dissolved by stomach acids to release an active agent.

Commercial acceptance of such devices and products is dependent on a variety of factors, such as cost to manufacture, shelf life or stability during storage, efficiency and/or timeliness of active agent delivery, biological capability, disposal issues, user comfort, controllability of release/delivery of active agents, and/or other factors. A device that addresses one or more of these factors is desirable.

### BRIEF SUMMARY

According to one aspect, an apparatus delivers active agents to a biological interface. The apparatus includes a first reservoir to contain a first ionic solution, a second reservoir to contain a second ionic solution, a deformable third reservoir to contain an active agent, and an activation device coupled to the first and second reservoirs in a manner to produce an ionic flow between the first and second ionic solutions and to further cause an osmotic solvent flow from the first reservoir to the second reservoir. The osmotic solvent flow into the second reservoir is capable to increase pressure inside the second reservoir in a manner that the increased pressure applies a compressive force to the third reservoir to expel at least some of the active agent contained therein.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a cross sectional block diagram of a device that can use electroosmotic flow to generate pressure to be applied to a reservoir so as to deliver an active agent to a biological interface according to one illustrated embodiment.

FIG. 2 is a cross sectional block diagram of the embodiment of the device of FIG. 1 showing compression of the reservoir and the resultant delivery of the active agent stored therein.

FIG. 3 is a cross sectional block diagram of an embodiment of a device having self-hydrating electrodes and that can use electroosmotic flow to generate pressure to be applied to a reservoir so as to deliver an active agent to a biological interface.

FIG. 4 is a cross sectional block diagram of an embodiment of a device that can be manually activated to generate pressure that can be applied to a reservoir so as to deliver an active agent to a biological interface.

FIG. 5 is a cross sectional block diagram of an embodiment of an iontophoresis device comprising active and counter electrode assemblies according to one illustrated embodiment where the active electrode assembly includes an outermost membrane caching an active agent, active agent adhered to an outer surface of the outermost membrane and a removable outer release liner overlying or covering the active agent and outermost membrane. One or more of the active agents can be delivered in response to pressure applied to a chamber and/or membrane containing the active agent(s).

FIG. 6 is a block diagram of the iontophoresis device of FIG. 5 positioned on a biological interface, with the outer release liner removed to expose the active agent according to one illustrated embodiment.

### DETAILED DESCRIPTION

As an overview, an embodiment of a device uses electroosmotic flow to assist in the delivery of an active agent, such as drugs, to a biological interface. The active agent is delivered from a flexible impermeable reservoir by compressing the reservoir. The compression is caused in one embodiment by electroosmotically pumping fluid into a chamber that surrounds the reservoir, thereby increasing the pressure in the chamber. The increased pressure in the chamber causes the reservoir to compress.

According to one embodiment, the electroosmotic pumping is generated by causing current to flow through two ionic solutions separated by an ion selective membrane. This current flow causes a concentration gradient to form across the ion selective membrane, and water will flow osmotically across the concentration gradient. A dedicated power source can be used in one embodiment to provide the current. In another embodiment, self-hydrating electrodes can be used to induce current flow, such as if the active agent is to be used for oral delivery. Alternatively or additionally to proving a power supply or other electrical stimulation, physical stimulation (such as finger pressure) can be used to cause compression of the reservoir.

The reservoir is provided with an outlet, such as a small needle, flexible catheter, or other orifice. When the reservoir is compressed, the active agent contained therein can exit through the outlet and into the biological interface adjacent to the outlet. Embodiments of the device can therefore be used to deliver an active agent to specific sites in a controlled manner, including temporal control of delivery by controlling the application of current by the power source.

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used herein and in the claims, the term “membrane” means a layer, barrier or material, which may, or may not be permeable. Unless specified otherwise, membranes may take the form a solid, liquid or gel, and may or may not have a distinct lattice or cross-linked structure.

As used herein and in the claims, the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein and in the claims, the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. Examples of commercially available cation exchange membranes include those available under the designators NEOSEPTA, CM-1, CM-2, CMX, CMS, and CMB from Tokuyama Co., Ltd. Examples of commercially available anion exchange membranes include those available under the designators NEOSEPTA, AM-1, AM-3, AMX, AHA, ACH and ACS also from Tokuyama Co., Ltd.

As used herein and in the claims, the term “bipolar membrane” means a membrane that is selective to two different charges or polarities. Unless specified otherwise, a bipolar membrane may take the form of a unitary membrane structure or multiple membrane structure. The unitary membrane structure may have a first portion including cation ion exchange material or groups and a second portion opposed to the first portion, including anion ion exchange material or groups. The multiple membrane structure (e.g., two film) may be formed by a cation exchange membrane attached or coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

A used herein and in the claims, the term “reservoir” means any form of mechanism to retain an element or compound in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound.

The headings provided herein are for convenience only and do not interpret the scope or meaning of the embodiments.

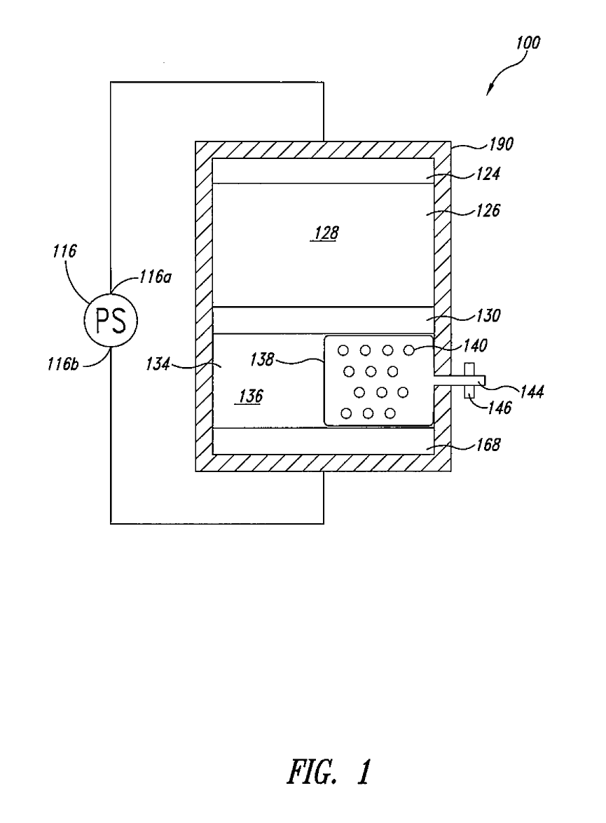

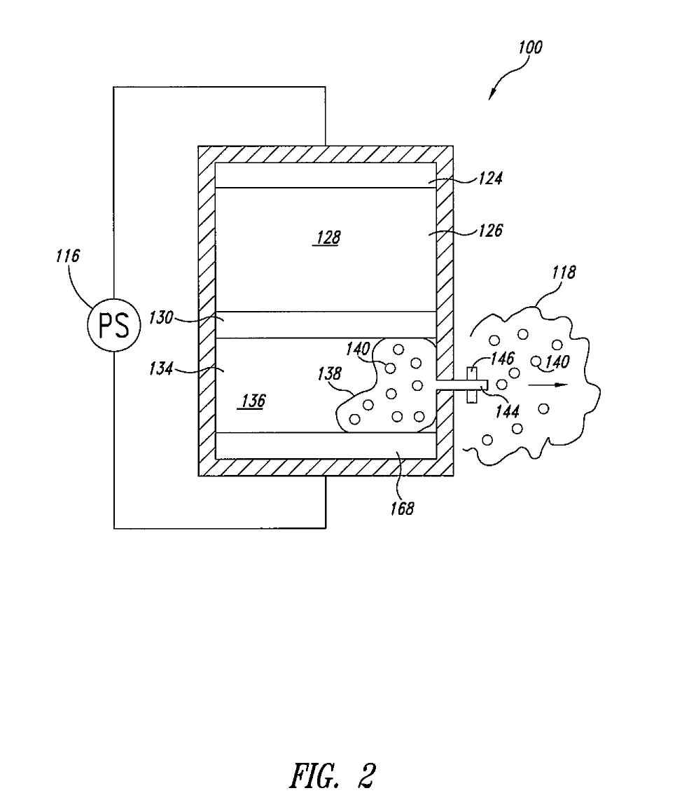

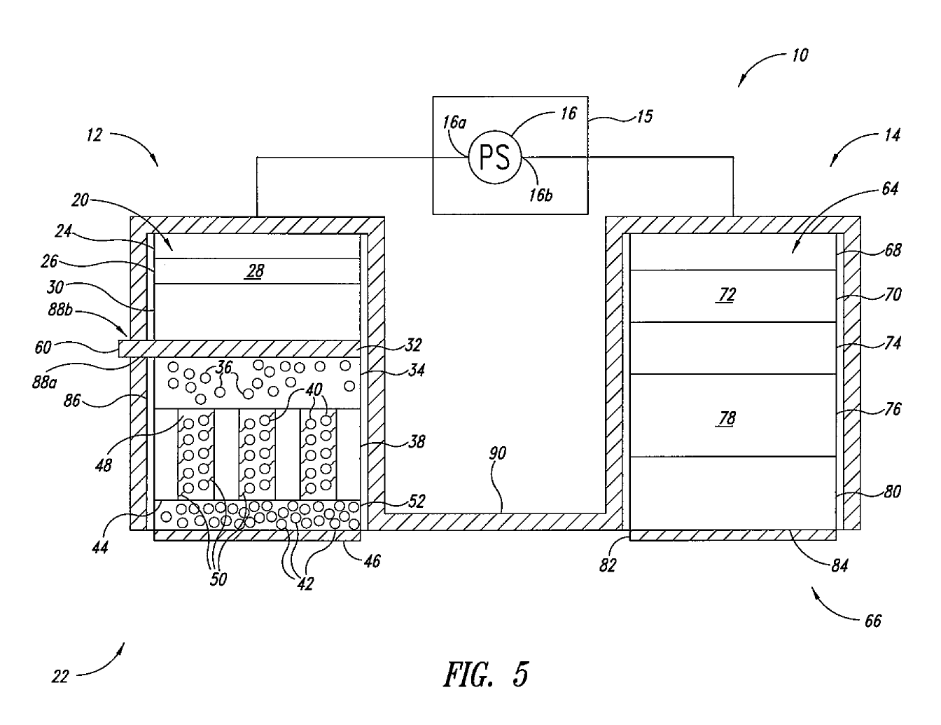

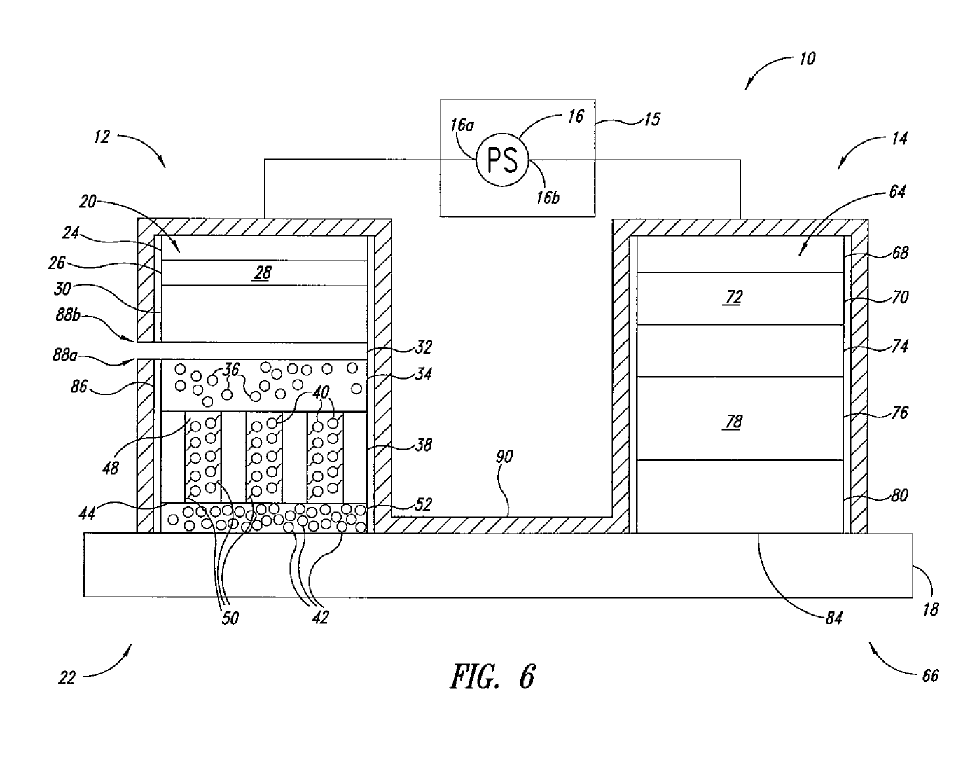

FIGS. 1 and 2 show an electroosmotic pump device 100 operable to supply an active agent to a biological interface 118 (FIG. 2), such as a portion of skin or mucous membrane, according to one illustrated embodiment. The device 100 comprises a power source 116 (or other activation device) coupled between a first electrode element 124 and a second electrode element 168. The power source 116 may or may not be included as part of a control unit 15, such as shown and described later with reference to FIGS. 5 and 6.

In the illustrated embodiment, the device 100 further comprises a first electrolyte reservoir 126 storing a first electrolyte 128, an inner ion selective membrane 130, a second electrolyte reservoir 134 storing a second electrolyte 136, a storage reservoir 138 contained inside the second electrolyte reservoir 126 to store an active agent 140, a delivery interface 144 coupled to the storage reservoir 138 to deliver the active agent 140 stored therein to the adjacent biological interface. According to an embodiment, the device 100 can also include a delivery control element 146 coupled to the delivery interface 144 to control the rate, timing, and/or quantity of the active agent 140 being delivered. A housing material 190 can be provided to encapsulate the various reservoirs and other elements of the device 100. Each of the above elements or structures will be discussed in detail below.

The first electrode element 124 is coupled to a first pole 116a of the power source 116 and positioned within the housing material 190 in a manner that an electromotive force or current or other controlled output waveform can be applied to transport electrolytes 128 and/or 136 across the inner ion selective membrane 130. The first electrode element 124 may take a variety of forms. For example, the first electrode element 124 may include a sacrificial element, for example a chemical compound or amalgam including silver (Ag) or silver chloride (AgCl). Such compounds or amalgams typically employ one or more heavy metals, for example lead (Pb), which may present issues with regard manufacturing, storage, use and/or disposal. Consequently, some embodiments may advantageously employ a carbon-based active electrode element 124. Such may, for example, comprise multiple layers, for example a polymer matrix comprising carbon and a conductive sheet comprising carbon fiber or carbon fiber paper, such as that described in commonly assigned pending Japanese Patent Application No. 2004/317317, filed Oct. 29, 2004.

The first electrolyte reservoir 126 may take a variety of forms including any structure capable of retaining the first electrolyte 128, and in some embodiments may even be the first electrolyte 128 itself, for example, where the first electrolyte 128 is in a gel, semi-solid or solid form. As another example, the first electrolyte reservoir 126 may take the form of a pouch or other receptacle, a membrane with pores, cavities or interstices, particularly where the first electrolyte 128 is a liquid. The first electrolyte reservoir 126 may or may not have a fixed volume (e.g., rigid containing walls).

The first electrolyte 128 of one embodiment can comprise salt (e.g., NaCl) dissolved in water according to a certain concentration. Alternatively or additionally, the first electrolyte 128 may comprise a substance identical or similar to the active agent that will be delivered. The first electrolyte 128 may provide ions or donate charges to prevent or inhibit the formation of gas bubbles (e.g., hydrogen) on the first electrode element 124 in order to enhance efficiency and/or increase delivery rates, or other purposes such as those described with reference to the electrolyte 28 of FIGS. 5-6.

The inner ion selective membrane 130 is generally positioned to separate the first electrolyte 128 and the second electrolyte reservoir 134 having the second electrolyte 136. The inner ion selective membrane 130 may take the form of a charge selective membrane. For example, the inner ion selective membrane 130 may take the form of an anion exchange membrane, selective to substantially pass anions and substantially block cations. Also, for example, the inner ion selective membrane 130 may take the form of a cationic exchange membrane, selective to substantially pass cations and substantially block anions. The inner ion selective membrane 130 may also, if desired, prevent transfer of undesirable elements or compounds between the first electrolyte reservoir 126 and the second electrolyte reservoir 134. Some embodiments may omit one or more or even all of the membranes described herein.

In another embodiment, the inner ion selective membrane 130 may take the form of a semi-permeable membrane that is substantially selective based on a size or molecular weight of the ion. In such an embodiment, the inner ion selective membrane 130 substantially passes ions of a first molecular weight or size, while substantially blocking passage of ions of a second molecular weight or size, greater than the first molecular weight or size.

The second electrolyte reservoir 134 is positioned on the opposite side of the inner ion selective membrane 130 from the first electrolyte reservoir 126. The second electrolyte reservoir 134 may take a variety of forms including any structure capable of having a fixed volume or a rigid structure that can contain the second electrolyte 136. For example, the second electrolyte reservoir 134 may take the form of a pouch with fixed volume or other receptacle; a membrane with pores, cavities or interstices; or any other type of structure that can have its internal pressure increased so as to apply compressive force to the storage reservoir 138.

The second electrolyte 136 of one embodiment can comprise a same electrolyte as the first electrolyte 128 but with a different concentration, such as NaCl dissolved in water at a different concentration. In another embodiment, the second electrolyte 136 can comprise a different electrolyte than the first electrolyte 128 at a same or different concentration.

The storage reservoir 138 of one embodiment is positioned inside the second electrolyte reservoir 134. The storage reservoir 138 can be made from a flexible material, such as a flexible rubber or plastic material, such that the volume of the storage reservoir 138 can be reduced in response to external compressive force. That is, since the volume of the second electrolyte reservoir 134 is fixed, an increase in the amount of solvent contained in the second electrolyte reservoir 134 will necessarily increase the internal pressure inside the second electrolyte reservoir 134. This increased internal pressure will press against the storage reservoir 138, thereby causing the storage reservoir 138 to compress (i.e., decrease its volume) by releasing some of its active agent 140 stored therein through the delivery interface 144.

In another embodiment, the storage reservoir 138 can be positioned externally to the second electrolyte reservoir 134. In such an embodiment, both the storage reservoir 138 and the second electrolyte reservoir 134 are made from a deformable material that can expand/contract, and both are contained within a common rigid structure. Therefore, when the second electrolyte reservoir 134 expands, the expansion will cause the storage reservoir 138 to contract, thereby causing expulsion of at least some of the active agent 140 stored therein.

Examples of the delivery interface 144 may include, but are not limited to, a mechanical valve structure, a porous membrane, a semi-permeable membrane, a charge selective membrane, a bipolar membrane, an orifice, a catheter, a small cannula, a material that dissolves or breaks in response to application of a pressure (such as pressure by the active agent 140 to exit from the storage reservoir 138) and/or breaks or dissolves in response to contact with the active agent 140 (or in response to contact with other material), one or more needles (including microneedles or other microstructures), or other structure capable to allow delivery of the active agent 140 whether in liquid, semi-solid, or solid form.

As stated above, the delivery interface 144 of one embodiment can comprise microneedles. Microneedles and microneedle arrays, their manufacture, and use have been described. Microneedles, either individually or in arrays, may be hollow; solid and permeable; solid and semi-permeable; or solid and non-permeable. Solid, non-permeable microneedles may further comprise grooves along their outer surfaces. Microneedle arrays, comprising a plurality of microneedles, may be arranged in a variety of configurations, for example rectangular or circular. Microneedles and microneedle arrays may be manufactured from a variety of materials, including silicon; silicon dioxide; molded plastic materials, including biodegradable or non-biodegradable polymers; ceramics; and metals. Microneedles, either individually or in arrays, may be used to dispense or sample fluids through the hollow apertures, through the solid permeable or semi-permeable materials, or via the external grooves. Microneedle devices are used, for example, to deliver a variety of compounds and compositions to the living body via a biological interface, such as skin or mucous membrane. In certain embodiments, the active agent compounds and compositions may be delivered into or through the biological interface. For example, in delivering compounds or compositions via the skin, the length of the microneedle(s), either individually or in arrays, and/or the depth of insertion may be used to control whether administration of a compound or composition is only into the epidermis, through the epidermis to the dermis, or subcutaneous. In certain embodiments, microneedle devices may be useful for delivery of high-molecular weight active agents, such as those comprising proteins, peptides and/or nucleic acids, and corresponding compositions thereof. In certain embodiments, for example wherein the fluid is an ionic solution, microneedle(s) or microneedle array(s) can provide electrical continuity between a power source and the tip of the microneedle(s). Microneedle(s) or microneedle array(s) may be used advantageously to deliver or sample compounds or compositions by electroosmotic and/or iontophoretic methods, as disclosed herein. In certain embodiments, for example, a plurality of microneedles in an array may advantageously be formed on an outermost biological interface-contacting surface of the device 100 and/or other devices disclosed herein. Compounds or compositions delivered or sampled by such a device may comprise, for example, high-molecular weight active agents, such as proteins, peptides and/or nucleic acids.

The delivery control element 146 may be provided, for instance, to control the rate, timing, and/or amount of the delivery of the active agent 140. For example, an embodiment of the delivery control element 146 can comprise a structure that prevents flow until a certain pressure is reached. As another example, the amount of flow allowed by the delivery control element 146 can be correspondingly adjusted (such allowing increased flow) based on the amount of pressure being applied by the active agent 140. The delivery control element 146 can be embodied as a mechanical structure, electromechanical structure, electrochemical structure, chemical structure (such as a closure made from a compound that dissolves at a certain rate to correspondingly increase an opening to allow passage of the active agent 140), and/or combination of such structures.

The second electrode element 168 allows completion of an electrical path between the poles 116a, 116b of the power source 116 via the first electrode element 124 and the other elements inside the housing material 190 of the device 100. The second electrode element 168 is electrically coupled to the second pole 116b of the power source 116, the second pole 116b having an opposite polarity to the first pole 116a. The second electrode element 168 may take a variety of forms suitable for closing the circuit by providing a return path. For example, the second electrode element 168 may include a sacrificial element, such as a chemical compound or amalgam including silver (Ag) or silver chloride (AgCl), or may include a non-sacrificial element such as the carbon-based electrode element discussed above.

The power source 116 may take the form of one or more chemical battery cells, super- or ultra-capacitors, or fuel cells. The power source 116 may, for example, provide a voltage of 12.8V DC, with tolerance of 0.8V DC, and a current of 0.3 mA. The power source 116 may be selectively electrically coupled to the first and second electrode elements 124, 168 via carbon fiber ribbons. Further, as suggested above, the first electrolyte 128 and the second electrolyte 136 may take the form of a cationic or an anionic compounds, including drugs or other therapeutic agent. Consequently, the terminals or poles 116a, 116b of the power source 116 may be reversed as appropriate. Likewise, the selectivity of the inner ion selective membrane 130 may be reversed. The particular polarity of the power source 116, the type and/or polarity and/or concentration of the first electrolyte 128 and the second electrolyte 136, and/or the type (e.g., charge selectivity or semi-permeability) of the inner ion selective membrane 130 can be chosen according to various embodiments, such that sufficient pressure due to electroosmosis flow can be increased in the second electrolyte reservoir 134 to compress the storage reservoir 138.

FIG. 2 illustrates an example operation of the device 100 according to an embodiment. In such an embodiment, the inner ion selective membrane 130 separates two ionic solutions in the first and second electrolyte reservoirs 126 and 134, but allows ions and water to pass through. The power source 116 generates a current that passes through the ionic solutions and the inner ion selective membrane 130, and solvent flow is induced.

In one example embodiment, the ionic solutions in the in the first and second electrolyte reservoirs comprise salt (such as NaCl) of different concentrations, with the inner ion selective membrane 130 being a cation or anion selective membrane. When the current is passed through the salt solutions and the ion selective membrane 130, a concentration gradient forms across the ion selective membrane 130 that is proportional to the amount of the current. As a result of the concentration gradient, water will flow osmotically across the concentration gradient (i.e., flow into the second electrolyte reservoir 134) in order to reach equilibrium in the concentration of the solutions.

Since the volume of the second electrolyte reservoir 134 is fixed, the pressure therein will rise, until equilibrium pressure is reached with the first electrolyte reservoir 126. This increasing pressure applies a compressive force against the storage reservoir 138, as depicted in FIG. 2, causing the storage reservoir 138 to reduce its volume by at least partially emptying its contents (i.e., the active agent 140).

With regards to a specific example where the first electrode element 124 and/or the second electrode element 168 are made from Ag or AgCl, the first electrode element 124 can comprise an anode, while the second electrode element comprises a cathode. When current is applied from the power source 116, Cl— combines at the anode to form AgCl and the reverse reaction occurs at the cathode. If the inner ion selective membrane 130 comprises a cation selective membrane, then water flows from the anode to the cathode and the resulting osmotic pressure will squeeze the storage reservoir 138.

The active agent 140 exits the storage reservoir 138 through the delivery interface 144 and into the surrounding biological interface 118. As explained above, the amount, timing, and/or rate of delivery of the active agent 140 can be controlled using the delivery control element 146.

Alternatively or additionally, the delivery of the active agent 140 can be controlled by controlling the application of current from the power source 116. For instance, through modulation, the shape or profile (such as duty cycle or waveform) of the applied current can be designed such that the current amplitude varies with time, thereby resulting in varying pressure applied to the storage reservoir 138. Examples of techniques to control the application of current are disclosed in U.S. Provisional Application Ser. No. 60/722,191, entitled “IONTOPHORESIS APPARATUS AND METHOD TO DELIVER ACTIVE AGENTS TO BIOLOGICAL INTERFACES USING A CAPACITIVE CIRCUIT,” filed Sep. 30, 2005; and in U.S. Provisional Patent Application Ser. No. 60/722,653, entitled “IONTOPHORESIS APPARATUS AND METHOD TO DELIVER ACTIVE AGENTS TO BIOLOGICAL INTERFACES USING MODULATED CURRENT TO REDUCE IRRITATION,” filed Sep. 30, 2005; both of which are assigned to the same assignee as the present application and incorporated herein by reference in their entireties.

The device 100 of FIGS. 1-2 can be embodied as a self-contained unit designed for oral ingestion, such as in pill form. Therefore, the biological interface 118 can comprise a stomach lining, intestinal lining, mucus membrane, or other internal surface inside the human body. In other embodiments, embodiments of the device 100 can be used externally. One example is use as a patch or other device to provide controlled release. Thus in such embodiment(s), the device 100 can be a permanent or temporary subdermal implant or externally attached device that provides subdermal delivery of the active agent 140.

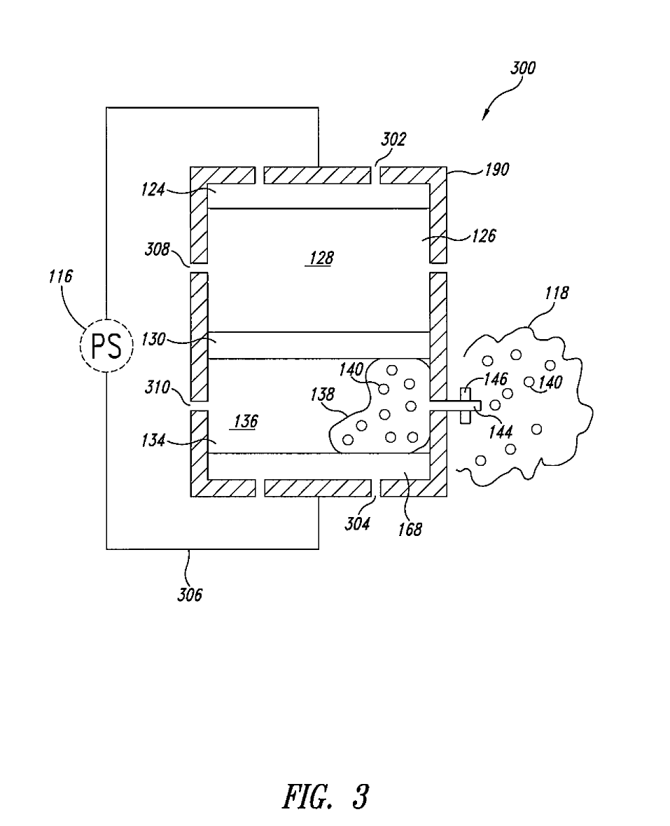

The embodiments of FIGS. 1-2 use the power source 116 to supply a current to induce electroosmotic solvent flow. FIG. 3 shows an embodiment of a device 300 that can produce electroosmotic power flow, without necessarily using (but can still use in another embodiment) the power supply 116. The device 300 is self-hydrating in that one or more elements inside the housing material 190 can be hydrated to create an ionic solution and/or a battery that generates current flow. Such hydration can occur, for example, if the device 300 is orally ingested by a patient along with water and/or by mixing with fluids inside the patient's body.

Specifically in one embodiment, the housing material 190 can be provided with a first set of orifices 302 to allow hydration of the first electrode element 124, and a second set of orifices 304 to allow hydration of the second electrode element 168. A conductor 306 may be provided for electrical coupling between the first electrode element 124 and the second electrode element 168, and/or a natural conductor can be provided by way of bodily fluids or hydrated tissue that can carry charge.

Prior to use of the device 300, the first electrode element 124 and the second electrode element 168 may be substantially inert, such as if they are in dry solid form. When placed in a hydrating environment, the hydrating fluid (such as water) enters the orifices 302 and 304 and transforms the first electrode element 124 and the second electrode element 168 into ionic solutions. If the first electrolyte reservoir 126 and the second electrolyte reservoir 134 are sufficiently hydrated, then current will flow through these reservoirs (via the ion exchange membrane 130), the first electrode element 124, the conductor 306, and the second electrode element 168.

In one embodiment, the first electrode element 124 and the second electrode element 168 can comprise zinc (Zn), copper (Cu), or other elements or compounds that can be arranged as a voltaic pile that can be hydrated to produce current flow. A person skilled in the art having the benefit of this disclosure can design such self-hydrating voltaic piles to produce current flow.

This current flow will generate the concentration gradient across the ion exchange membrane 130, which in turn will cause osmotic water flow into the second electrolyte reservoir 134. The osmotic water flow into the second electrolyte reservoir 134 will compress the storage reservoir 138, thereby causing the active agent 140 to be released into the surrounding biological interface 118.

As stated above, the power source 116 may or may not be provided in the embodiment of the device 300, and therefore, the power source 116 is depicted as broken lines in FIG. 3. The power source 116 can be provided in some embodiments of the device 300, for example, where additional electromotive force may be desired.

In some embodiments of the device 300, the first electrolyte 128 in the first electrolyte reservoir 126 and/or the second electrolyte 136 in the second electrolyte reservoir 134 may not be hydrated or otherwise sufficiently dissolved prior to use of the device 300. Accordingly, the housing material 190 may be provided with a third set of orifices 308 for the first electrolyte reservoir 126 and/or a second set of orifices 310 for the second electrolyte reservoir 134, to allow solvent (such as water) to enter these respective reservoirs to hydrate the electrolytes contained therein, when the device 300 is placed into use.

According to one embodiment, either one or both of the electrode elements may not be hydrated, while both of the electrolytes are hydrated, prior to use of the device 300. In another embodiment, one or both of the electrode elements may not be hydrated, while one or both of the electrolytes are hydrated, prior to use of the device 300. In still another embodiment, none of the electrode elements and electrolytes are hydrated prior to use of the device 300. The number and location of the various sets of orifices of the housing material 190 can be designed according to which particular element of the device 300 is to be hydrated when the device 300 is put into use.

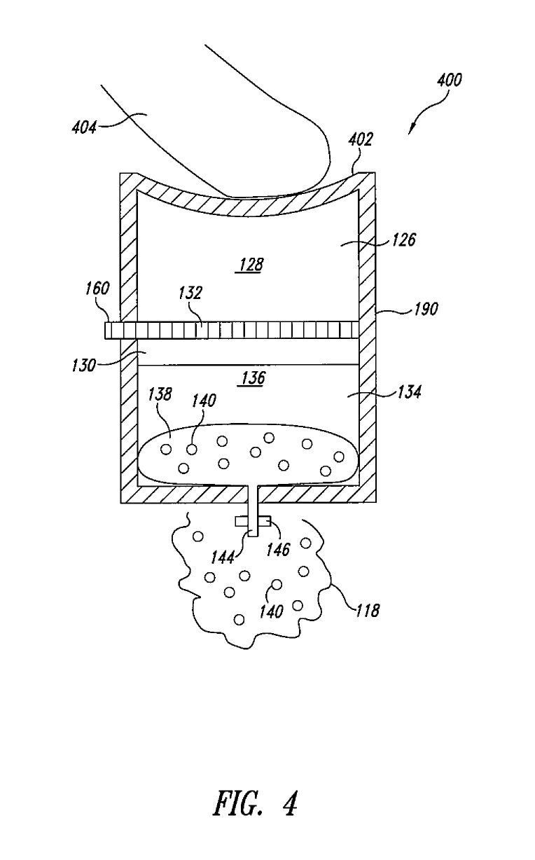

FIG. 4 illustrates an embodiment of a device 400 that can use osmotic flow to generate pressure to deliver the active agent 140, without using electrical stimulation, such as from a dedicated power source 116. In such an embodiment, external pressure is applied to a non-resilient or otherwise deformable portion 402 of the housing material 190.

In the example of FIG. 4, the external pressure is provided by a fingertip 404, such as a fingertip of the patient or medical personnel. The fingertip 404 deforms the portion 402 inwardly, so as to induce ionic flow between the first electrolyte reservoir 126 and the second electrolyte reservoir 134. This ionic flow will also cause a corresponding osmotic fluid flow, which will in turn cause compression of the storage reservoir 138 to release the active agent 140 into the surrounding biological interface 118.

Alternatively or additionally to inducing ionic flow, the pressure from the fingertip 404 can also cause reactive pressures within the various reservoirs inside the housing material 190, which in turn will cause the storage reservoir 138 to compress and expel the active agent 140. The pressure from the fingertip 404 can be applied and released, applied continuously to press and hold down on the portion 402, and/or applied intermittently. The degree and duration of the pressure applied from the fingertip 404 can vary from one embodiment to the other based on factors such as the type and quantity of active agent 140 to be delivered, the timing of delivery, the type and concentration of the first electrolyte 128 and/or the second electrolyte 136, the elasticity of the portion 402, and so forth.

In one embodiment, an inner sealing liner 132 can be provided to separate the first electrolyte reservoir 126 from the second electrolyte reservoir 134, prior to using the device 400. The inner sealing liner 132 may advantageously prevent migration or diffusion between the first electrolyte reservoir 126 and the second electrolyte reservoir 134, during storage for example. When the device 400 is ready for use and prior to applying the external pressure to the portion 402, the inner sealing liner 132 can be removed by pulling on an external tab 160.

FIGS. 5 and 6 show an iontophoresis device 10 that can operate to deliver one or more active agents in response to electromotive stimulation and also in response to electroosmotic fluid flow/pressure in a manner similar to some of the embodiments described above. The iontophoresis device 10 of one embodiment comprises active and counter electrode assemblies, 12, 14, respectively, electrically coupled to a control unit 15 having a power source 16, operable to supply an active agent to a biological interface 18 (FIG. 6), such as a portion of skin or mucous membrane via iontophoresis, according to one illustrated embodiment. The control unit 15 can operate, for example, to provide a controlled output waveform for inducing ionic flow (and therefore osmotic flow) within the reservoirs of the iontophoresis device 10.

In the illustrated embodiment, the active electrode assembly 12 comprises, from an interior 20 to an exterior 22 of the active electrode assembly 12, an active electrode element 24, an electrolyte reservoir 26 storing an electrolyte 28, an inner ion selective membrane 30, an inner sealing liner 32, an inner active agent reservoir 34 storing active agent 36, an outermost ion selective membrane 38 that caches additional active agent 40, further active agent 42 carried by an outer surface 44 of the outermost ion selective membrane 38, and an outer release liner 46. Each of the above elements or structures will be discussed in detail below.

The active electrode element 24 is coupled to a first pole 16a of the power source 16 and positioned in the active electrode assembly 12 in a manner that an electromotive force or current or other controlled output waveform can be applied to transport active agent 36, 40, 42 via various other components of the active electrode assembly 12. The active electrode element 24 may take a variety of forms. For example, the active electrode element 24 may include a sacrificial element, for example a chemical compound or amalgam including silver (Ag) or silver chloride (AgCl). Such compounds or amalgams typically employ one or more heavy metals, for example lead (Pb), which may present issues with regard manufacturing, storage, use and/or disposal. Consequently, some embodiments may advantageously employ a carbon-based active electrode element 24. Such may, for example, comprise multiple layers, for example a polymer matrix comprising carbon and a conductive sheet comprising carbon fiber or carbon fiber paper, such as that described in commonly assigned pending Japanese Patent Application No. 2004/317317, filed Oct. 29, 2004.

In use, the outermost active electrode ion selective membrane 38 may be placed directly in contact with the biological interface 18 (FIG. 6). Alternatively, an interface-coupling medium (not shown) may be employed between the outermost active electrode ion selective membrane 38 and the biological interface 18. The interface-coupling medium may, for example, take the form of an adhesive and/or gel. The gel may, for example, take the form of a hydrating gel or a hydrogel. If used, the interface-coupling medium should be permeable by any one or more of the active agents 36, 40, 42.

The electrolyte reservoir 26 may take a variety of forms including any structure capable of retaining electrolyte 28, and in some embodiments may even be the electrolyte 28 itself, for example, where the electrolyte 28 is in a gel, semi-solid or solid form. For example, the electrolyte reservoir 26 may take the form of a pouch or other receptacle, a membrane with pores, cavities or interstices, particularly where the electrolyte 28 is a liquid.

The electrolyte 28 may provide ions or donate charges to prevent or inhibit the formation of gas bubbles (e.g., hydrogen) on the active electrode element 24 in order to enhance efficiency and/or increase delivery rates. This elimination or reduction in electrolysis may in turn inhibit or reduce the formation of acids and/or bases (e.g., H⁺ ions, OH⁻ ions), that would otherwise present possible disadvantages such as reduced efficiency, reduced transfer rate, and/or possible irritation of the biological interface 18. As discussed further below, in some embodiments the electrolyte 28 may provide or donate ions to substitute for the active agent, for example substituting for the active agent 40 cached thereon. Such may facilitate transfer of the active agent 40 to the biological interface 18, for example, increasing and/or stabilizing delivery rates. A suitable electrolyte may take the form of a solution of 0.5M disodium fumarate: 0.5M Poly acrylic acid (5:1).

The inner ion selective membrane 30 is generally positioned to separate the electrolyte 28 and the inner active agent reservoir 34. The inner ion selective membrane 30 may take the form of a charge selective membrane. For example, where the active agent 36, 40, 42 comprises a cationic active agent, the inner ion selective membrane 38 may take the form of an anion exchange membrane, selective to substantially pass anions and substantially block cations. Also, for example, where the active agent 36, 40, 42 comprises an anionic active agent, the inner ion selective membrane 38 may take the form of an cationic exchange membrane, selective to substantially pass cations and substantially block anions. The inner ion selective membrane 38 may advantageously prevent transfer of undesirable elements or compounds between the electrolyte 28 and the active agents 26, 40, 42. For example, the inner ion selective membrane 38 may prevent or inhibit the transfer of hydrogen (H⁺) or sodium (Na⁺) ions from the electrolyte 72, which may increase the transfer rate and/or biological compatibility of the iontophoresis device 10.

The inner sealing liner 32 separates the active agent 36, 40, 42 from the electrolyte 28 and is selectively removable, as discussed in detail below with respect to FIG. 2. The inner sealing liner 32 may advantageously prevent migration or diffusion between the active agent 36, 40, 42 and the electrolyte 28, for example, during storage.

The inner active agent reservoir 34 is generally positioned between the inner ion selective membrane 30 and the outermost ion selective membrane 38. The inner active agent reservoir 34 may take a variety of forms including any structure capable of temporarily retaining active agent 36, and in some embodiments may even be the active agent 36 itself, for example, where the active agent 36 is in a gel, semi-solid or solid form. For example, the inner active agent reservoir 34 may take the form of a pouch or other receptacle, a membrane with pores, cavities or interstices, particularly where the active agent 36 is a liquid. The inner active agent reservoir 34 may advantageously allow larger doses of the active agent 36 to be loaded in the active electrode assembly 12.

The outermost ion selective membrane 38 is positioned generally opposed across the active electrode assembly 12 from the active electrode element 24. The outermost membrane 38 may, as in the embodiment illustrated in FIGS. 5 and 6, take the form of an ion exchange membrane, pores 48 (only one called out in FIGS. 5 and 6 for sake of clarity of illustration) of the ion selective membrane 38 including ion exchange material or groups 50 (only three called out in FIGS. 5 and 6 for sake of clarity of illustration). Under the influence of an electromotive force or current, the ion exchange material or groups 50 selectively substantially passes ions of the same polarity as active agent 36, 40, while substantially blocking ions of the opposite polarity. Thus, the outermost ion exchange membrane 38 is charge selective. Where the active agent 36, 40, 42 is a cation (e.g., lidocaine), the outermost ion selective membrane 38 may take the form of a cation exchange membrane. Alternatively, where the active agent 36, 40, 42 is an anion, the outermost ion selective membrane 38 may take the form of an anion exchange membrane.

The outermost ion selective membrane 38 may advantageously cache active agent 40. In particular, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

Alternatively, the outermost ion selective membrane 38 may take the form of semi-permeable or microporous membrane which is selective by size. In some embodiments, such a semi-permeable membrane may advantageously cache active agent 40, for example by employing the removably releasable outer release liner 46 to retain the active agent 40 until the outer release liner 46 is removed prior to use.

The outermost ion selective membrane 38 may be preloaded with the additional active agent 40, such as ionized or ionizable drugs or therapeutic agents and/or polarized or polarizable drugs as the therapeutic agents. Where the outermost ion selective membrane 38 is an ion exchange membrane, a substantial amount of active agent 40 may bond to ion exchange groups 50 in the pores, cavities or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness so as to form a distinct layer 52. In other embodiments, the further active agent 42 may not be sufficient in volume, thickness or coverage as to constitute a layer in a conventional sense of such term.

The active agent 42 may be deposited in a variety of highly concentrated forms such as, for example, solid form, nearly saturated solution form or gel form. If in solid form, a source of hydration may be provided, either integrated into the active electrode assembly 12, or applied from the exterior thereof just prior to use.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. Thus, a first type of active agent may be stored in the inner active agent reservoir 34, while a second type of active agent may be cached in the outermost ion selective membrane 38. In such an embodiment, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 36, 40, 42 will all be of common polarity to prevent the active agents 36, 40, 42 from competing with one another. Other combinations are possible.

The outer release liner 46 may generally be positioned overlying or covering further active agent 42 carried by the outer surface 44 of the outermost ion selective membrane 38. The outer release liner 46 may protect the further active agent 42 and/or outermost ion selective membrane 38 during storage, prior to application of an electromotive force or current. The outer release liner 46 may be a selectively releasable liner made of waterproof material, such as release liners commonly associated with pressure sensitive adhesives. Note that the inner release liner 46 is shown in place in FIG. 5 and removed in FIG. 6.

The counter electrode assembly 14 allows completion of an electrical path between poles 16a, 16b of the power source 16 via the active electrode assembly 12 and the biological interface 18. The counter electrode assembly 14 may take a variety of forms suitable for closing the circuit by providing a return path.

In the embodiment illustrated in FIGS. 5 and 6 the counter electrode assembly comprises, in order from an interior 64 to an exterior 66 of the counter electrode assembly 14: a counter electrode element 68, electrolyte reservoir 70 storing an electrolyte 72, an inner ion selective membrane 74, an optional buffer reservoir 76 storing buffer material 78, an outermost ion selective membrane 80, and an outer release liner 82.

The counter electrode element 68 is electrically coupled to a second pole 16b of the power source 16, the second pole 16b having an opposite polarity to the first pole 16a. The counter electrode element 68 may take a variety of forms. For example, the counter electrode element 68 may include a sacrificial element, such as a chemical compound or amalgam including silver (Ag) or silver chloride (AgCl), or may include a non-sacrificial element such as the carbon-based electrode element discussed above.

The electrolyte reservoir 70 may take a variety of forms including any structure capable of retaining electrolyte 72, and in some embodiments may even be the electrolyte 72 itself, for example, where the electrolyte 72 is in a gel, semi-solid or solid form. For example, the electrolyte reservoir 70 may take the form of a pouch or other receptacle, or a membrane with pores, cavities or interstices, particularly where the electrolyte 72 is a liquid.

The electrolyte 72 is generally positioned between the counter electrode element 68 and the outermost ion selective membrane 80, proximate the counter electrode element 68. The electrolyte 72 may provide ions or donate charges to prevent or inhibit the formation of gas bubbles (e.g., hydrogen) on the counter electrode element 68 and may prevent or inhibit the formation of acids or bases or neutralize the same, which may enhance efficiency and/or reduce the potential for irritation of the biological interface 18.

The inner ion selective membrane 74 is positioned between and/or to separate, the electrolyte 72 from the buffer material 78. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydrogen (H⁺) or sodium (Na⁺) ions from the electrolyte 72 into the buffer material 78.

The optional buffer reservoir 76 is generally disposed between the electrolyte reservoir and the outermost ion selective membrane 80. The buffer reservoir 76 may take a variety of forms capable of temporarily retaining the buffer material 78. For example, the buffer reservoir 76 may take the form of a cavity, a porous membrane or a gel.

The buffer material 78 may supply ions for transfer through the outermost ion selective membrane 42 to the biological interface 18. Consequently, the buffer material 78 may, for example, comprise a salt (e.g., NaCl).

The outermost ion selective membrane 80 of the counter electrode assembly 14 may take a variety of forms. For example, the outermost ion selective membrane 80 may take the form of a charge selective ion exchange membrane, such as a cation exchange membrane or an anion exchange membrane, which substantially passes and/or blocks ions based on the charge carried by the ion. Examples of suitable ion exchange membranes are discussed above. Alternatively, the outermost ion selective membrane 80 may take the form of a semi-permeable membrane that substantially passes and/or blocks ions based on size or molecular weight of the ion.

The outermost ion selective membrane 80 of the counter electrode assembly 14 is selective to ions with a charge or polarity opposite to that of the outermost ion selective membrane 38 of the active electrode assembly 12. Thus, for example, where the outermost ion selective membrane 38 of the active electrode assembly 12 allows passage of negatively charged ions of the active agent 36, 40, 42 to the biological interface 18, the outermost ion selective membrane 80 of the counter electrode assembly 14 allows passage of positively charged ions to the biological interface 18, while substantially blocking passage of ions having a negative charge or polarity. On the other hand, where the outermost ion selective membrane 38 of the active electrode assembly 12 allows passage of positively charged ions of the active agent 36, 40, 42 to the biological interface 18, the outermost ion selective membrane 80 of the counter electrode assembly 14 allows passage of negatively charged ions to the biological interface 18 while substantially blocking passage of ions with a positive charge or polarity.

The outer release liner 82 may generally be positioned overlying or covering an outer surface 84 of the outermost ion selective membrane 80. Note that the inner release liner 82 is shown in place in FIG. 5 and removed in FIG. 6. The outer release liner 82 may protect the outermost ion selective membrane 80 during storage, prior to application of an electromotive force or current. The outer release liner 82 may be a selectively releasable liner made of waterproof material, such as release liners commonly associated with pressure sensitive adhesives. In some embodiments, the outer release liner 82 may be coextensive with the outer release liner 46 of the active electrode assembly 12.

The power source 16 may take the form of one or more chemical battery cells, super- or ultra-capacitors, or fuel cells. The power source 16 may, for example, provide a voltage of 12.8V DC, with tolerance of 0.8V DC, and a current of 0.3 mA. The power source 16 may be selectively electrically coupled to the active and counter electrode assemblies 12, 14 via circuitry in the control unit 15, for example, via carbon fiber ribbons. The control unit 15 of the iontophoresis device 10 may include discrete and/or integrated circuit elements to control the voltage, current, and/or power delivered to the electrode assemblies 12, 14. For example, the iontophoresis device 10 may include a diode to control the output signal provided to the electrode elements 20, 68 and/or may include other elements to control a characteristic of the output signal used to transfer any one or more of the active agent 36, 40, 42 to the biological interface 18. Embodiments of the control unit 15 that provide a controlled output signal using a capacitive circuit will be described later below.

As suggested above, the active agent 36, 40, 42 may take the form of a cationic or an anionic drug or other therapeutic agent. Consequently, the terminals or poles 16a, 16b of the power source 16 may be reversed. Likewise, the selectivity of the outermost ion selective membranes 38, 80 and inner ion selective membranes 30, 74 may be reversed.

The iontophoresis device 10 may further comprise an inert molding material 86 adjacent exposed sides of the various other structures forming the active and counter electrode assemblies 12, 14. The molding material 86 may advantageously provide environmental protection to the various structures of the active and counter electrode assemblies 12, 14. Molding material 86 may form a slot or opening 88a on one of the exposed sides through which the tab 60 extends to allow for the removal of inner sealing liner 32 prior to use. Enveloping the active and counter electrode assemblies 12, 14 is a housing material 90. The housing material 90 may also form a slot or opening 88b positioned aligned with the slot or opening 88a in molding material 86 through which the tab 60 extends to allow for the removal of inner sealing liner 32 prior to use of the iontophoresis device 10.

Immediately prior to use, the iontophoresis device 10 is prepared by withdrawing the inner sealing liner 32 and removing the outer release liners 46, 82. As described above, the inner sealing liner 32 may be withdrawn by pulling on tab 60. The outer release liners 46, 82 may be pulled off in a similar fashion to remove release liners from pressure sensitive labels and the like.

As best seen in FIG. 6, the active and counter electrode assemblies 12, 14 are positioned on the biological interface 18. Positioning on the biological interface may close the circuit, allowing electromotive force to be applied and/or current to flow from one pole 16a of the power source 16 to the other pole 16b, via the active electrode assembly, biological interface 18 and counter electrode assembly 14.

In the presence of the electromotive force and/or current, active agent 36 is transported toward the biological interface 18. Additional active agent 40 is released by the ion exchange groups or material 50 by the substitution of ions of the same charge or polarity (e.g., active agent 36), and transported toward the biological interface 18. While some of the active agent 36 may substitute for the additional active agent 40, some of the active agent 36 may be transferred through the outermost ion elective membrane 38 into the biological interface 18. Further active agent 42 carried by the outer surface 44 of the outermost ion elective membrane 38 is also transferred to the biological interface 18.

During iontophoresis, the electromotive force across the electrode assemblies, as described, leads to a migration of charged active agent molecules, as well as ions and other charged components, through the biological interface into the biological tissue. This migration may lead to an accumulation of active agents, ions, and/or other charged components within the biological tissue beyond the interface. During iontophoresis, in addition to the migration of charged molecules in response to repulsive forces, there is also an electroosmotic flow of solvent (e.g., water) through the electrodes and the biological interface into the tissue. In certain embodiments, the electroosmotic solvent flow enhances migration of both charged and uncharged molecules. Enhanced migration via electroosmotic solvent flow may occur particularly with increasing size of the molecule.

The embodiments described above with reference to FIGS. 5-6 generally describe delivery of one or more active agents 36, 40, 42 to the biological interface 18 using electromotive force to drive these active agents to the biological interface 18. The delivery of one or more of these active agents 36, 40, 42 or other active agents may also be performed in an embodiment of the device 10 using the electroosmotic solvent flow described above.

For example, the active agents 40 and/or 42 may be contained in a deformable reservoir. The deformable reservoir can comprise a container made from a plastic or rubber or other non-resilient material, a gel, a compressable material impregnated with an active agent, or some other structure that is responsive to applied pressure to release the active agent store therein. The membrane 38 and/or the outer surface 44 described above can serve as the deformable reservoirs in one embodiment.

Thus, when osmotic flow increases pressure externally to the membrane 38 and/or to the outer surface 44 (such as increased pressure within the membrane 34), the increased pressure will force the active agents 40 and 42 contained therein towards the biological interface 18. The migration of the active agents 40 and 42 may further be assisted by the electromotive power provided by the power source 16.

In certain embodiments, the active agent may be a higher molecular weight molecule. In certain aspects, the molecule may be a polar polyelectrolyte. In certain other aspects, the molecule may be lipophilic. In certain embodiments, such molecules may be charged, may have a low net charge, or may be uncharged under the conditions within the active electrode. In certain aspects, such active agents may migrate poorly under the iontophoretic repulsive forces, in contrast to the migration of small more highly charged active agents under the influence of these forces. These higher molecular active agents may thus be carried through the biological interface into the underlying tissues primarily via electroosmotic solvent flow and/or through pressure caused by electroosmotic solvent flow as described above. In certain embodiments, the high molecular weight polyelectrolytic active agents may be proteins, polypeptides or nucleic acids.

The above description of illustrated embodiments, including what is described in the Abstract, is not intended to be exhaustive or to limit the claims to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to other agent delivery systems and devices, not necessarily the illustrative delivery devices generally described above. For instance, while some embodiments may include all of the membranes, reservoirs and other structures discussed above, other embodiments may omit some of the membranes, reservoirs or other structures, or may include additional structures. Also for example, some embodiments may include an interface layer interposed between the outermost active electrode ion selective membrane 22 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240995, herein incorporated in their entirety by reference. Further examples of such hydrogels are disclosed in U.S. Patent applications 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

In certain embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; an active agent reservoir (which may be deformable) having an active agent solution that is in contact with the first electrode member and to which is applied a voltage and/or current via the first electrode member; a biological interface contact member, which may be a microneedle array and is placed against the forward surface of the active agent reservoir; and a first cover or container that accommodates these members. The counter electrode assembly includes the following: a second electrode member connected to a negative electrode of the power source; an electrolyte reservoir that holds an electrolyte that is in contact with the second electrode member and to which voltage and/or current is applied via the second electrode member; and a second cover or container that accommodates these members.

In certain other embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; a first electrolyte reservoir having an electrolyte that is in contact with the first electrode member and to which is applied a voltage and/or current via the first electrode member; a first anion-exchange membrane that is placed on the forward surface of the first electrolyte reservoir; an active agent reservoir (which may be deformable) that is placed against the forward surface of the first anion-exchange membrane; a biological interface contacting member, which may be a microneedle array and is placed against the forward surface of the active agent reservoir; and a first cover or container that accommodates these members. The counter electrode assembly includes the following: a second electrode member connected to a negative electrode of the power source; a second electrolyte reservoir having an electrolyte that is in contact with the second electrode member and to which is applied a voltage and/or current via the second electrode member; a cation-exchange membrane that is placed on the forward surface of the second electrolyte reservoir; a third electrolyte reservoir that is placed against the forward surface of the cation-exchange membrane and holds an electrolyte to which a voltage and/or current is applied from the second electrode member via the second electrolyte reservoir and the cation-exchange membrane; a second anion-exchange membrane placed against the forward surface of the third electrolyte reservoir; and a second cover or container that accommodates these members.

Certain details of microneedle devices, their use and manufacture, are disclosed in U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; 6,939,311; all of which are incorporated herein by reference in their entirety. Some or all of the teaching therein may be applied to microneedle devices, their manufacture, and their use in iontophoretic applications.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: Japanese Patent Application Serial No. H03-86002, filed Mar. 27, 1991, having Japanese Publication No. H04-297277, issued on Mar. 3, 2000 as Japanese Patent No. 3040517; Japanese Patent Application Serial No. 11-033076, filed Feb. 10, 1999, having Japanese Publication No. 2000-229128; Japanese Patent Application Serial No. 11-033765, filed Feb. 12, 1999, having Japanese Publication No. 2000-229129; Japanese Patent Application Serial No. 11-041415, filed Feb. 19, 1999, having Japanese Publication No. 2000-237326; Japanese Patent Application Serial No. 11-041416, filed Feb. 19, 1999, having Japanese Publication No. 2000-237327; Japanese Patent Application Serial No. 11-042752, filed Feb. 22, 1999, having Japanese Publication No. 2000-237328; Japanese Patent Application Serial No. 11-042753, filed Feb. 22, 1999, having Japanese Publication No. 2000-237329; Japanese Patent Application Serial No. 11-099008, filed Apr. 6, 1999, having Japanese Publication No. 2000-288098; Japanese Patent Application Serial No. 11-099009, filed Apr. 6, 1999, having Japanese Publication No. 2000-288097; PCT Patent Application WO 2002JP4696, filed May 15, 2002, having PCT Publication No WO03037425; U.S. patent application Ser. No. 10/488,970, filed Mar. 9, 2004; Japanese patent application 2004/317317, filed Oct. 29, 2004; U.S. Provisional Patent Application Ser. No. 60/627,952, filed Nov. 16, 2004; Japanese Patent Application Serial No. 2004-347814, filed Nov. 30, 2004; Japanese Patent Application Serial No. 2004-357313, filed Dec. 9, 2004; Japanese Patent Application Serial No. 2005-027748, filed Feb. 3, 2005; and Japanese Patent Application Serial No. 2005-081220, filed Mar. 22, 2005.

Aspects of the various embodiments can be modified, if necessary, to employ systems, circuits and concepts of the various patents, applications and publications to provide yet further embodiments. While some embodiments may include all of the membranes, reservoirs and other structures discussed above, other embodiments may omit some of the membranes, reservoirs or other structures. Still other embodiments may employ additional ones of the membranes, reservoirs and structures generally described above. Even further embodiments may omit some of the membranes, reservoirs and structures described above while employing additional ones of the membranes, reservoirs and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

What is claimed is:

1. An apparatus to deliver active agents to a biological interface, the apparatus comprising:
a first reservoir to contain a first ionic solution;
a second reservoir to contain a second ionic solution;
a deformable third reservoir to contain an active agent; and
an activation device coupled to the first and second reservoirs in a manner to produce an ionic flow between the first and second ionic solutions and to further cause an osmotic solvent flow from the first reservoir to the second reservoir, the osmotic solvent flow into the second reservoir being capable to increase pressure inside the second reservoir in a manner that the increased pressure applies a compressive force to the third reservoir to expel at least some of the active agent contained therein.

2. The apparatus of claim 1 wherein the third reservoir is positioned inside of the second reservoir.

3. The apparatus of claim 1 wherein both the second and third reservoirs are deformable and are contained within a common rigid structure.

4. The apparatus of claim 1, further comprising an ion exchange membrane between the first and second reservoirs.

5. The apparatus of claim 1, further comprising:
a first electrode element positioned adjacent to the first reservoir;
a second electrode element positioned adjacent to the second reservoir; and
a conductor to electrically couple the first electrode element to the second electrode element.

6. The apparatus of claim 5 wherein the activation device comprises a power source coupled between the first and second electrode elements to provide an electromotive force to induce the ionic flow between the first and second ionic solutions.

7. The apparatus of claim 5 wherein the first and second electrode elements respectively comprise first and second hydratable electrode elements.

8. The apparatus of claim 7, further comprising a housing material to at least partially encapsulate the first and second hydratable electrode elements, the housing material having first and second sets of orifices to allow a hydrating fluid to enter the housing material to hydrate the first and second hydratable electrode elements.

9. The apparatus of claim 8 wherein the housing material has at least a third set of orifices to allow hydrating fluid to hydrate an electrolyte contained in the first reservoir or in the second reservoir.

10. The apparatus of claim 1, further comprising a housing material to at least partially encapsulate the first reservoir, wherein the activation element comprises a deformable portion of the housing material that is responsive to an external pressure to cause application of an internal pressure to compress the third reservoir.

11. The apparatus of claim 1, further comprising a delivery interface coupled to the third reservoir to deliver at least some of the active agent stored therein to a biological interface.

12. The apparatus of claim 11 wherein the delivery interface comprises one of a mechanical valve structure, a porous membrane, a semi-permeable membrane, a charge selective membrane, a bipolar membrane, an orifice, a catheter, a cannula, a material that loses structure in response to application of a pressure or in response to contact with the active agent, at least one needle, a microstructure, or an orifice.

13. The apparatus of claim 11, further comprising a delivery control element coupled to the delivery interface to control a rate or an amount of the active agent that is delivered to the biological interface.

14. The apparatus of claim 1 wherein the third reservoir comprises part of a pill that can be orally ingested, external patch, implantable device, iontophoresis device, or external device.

15. A method to deliver an active agent to a biological interface using electroosmosis, the method comprising:
providing a first ionic solution in a first reservoir;
providing a second ionic solution in a second reservoir;
providing an active agent in a third reservoir;
creating an osmotic flow of solvent from the first reservoir to the second reservoir; and
compressing the third reservoir to expel at least some of the active agent provided therein, in response to an increase in pressure in the second reservoir caused by the osmotic flow of solvent.

16. The method of claim 15, further comprising:
generating an ionic flow between the first and second reservoirs;
generating a concentration gradient between the first and second ionic solution; and
responding to the concentration gradient by creating the osmotic flow to generate an equilibrium state.

17. The method of claim 16 wherein generating the ionic flow comprises applying a current to the first and second ionic solutions.

18. The method of claim 16 wherein applying the electromotive force comprises applying the current comprises applying an electromotive force from a power supply.

19. The method of claim 17 wherein applying the current comprises hydrating a voltaic pile.

20. The method of claim 15 wherein creating the osmotic flow comprises applying an external pressure to the first reservoir to deform the first reservoir.

21. A system to deliver an active agent to a biological interface using electroosmosis, the system comprising:
first reservoir means for containing a first ionic solution;
second reservoir means for containing a second ionic solution;
third reservoir means for containing an active agent; and
activation means for creating an osmotic flow of solvent from the first reservoir means to the second reservoir means in a manner that compresses the third reservoir means to expel at least some of the active agent contained therein, in response to an increase in pressure in the second reservoir means caused by the osmotic flow of solvent.

22. The system of claim 21 wherein the activation means comprises power supply means for generating electromotive force to be applied to the first and second ionic solutions.

23. The system of claim 21 wherein the activation means comprises hydratable electrode means for producing current flow through the first and second ionic solutions.

24. The system of claim 21 wherein the activation means comprises a deformable portion means of a housing for transferring an external force to the first reservoir means.

25. The system of claim 21 wherein at least the third reservoir means comprises part of an iontophoresis device means for delivering the active agent to a biological interface.

26. The system of claim 21 wherein at least the third reservoir means comprises part of a pill means for orally ingesting the active agent, external patch means for externally applying the active agent, implantable device means for internally applying the active agent, or external device means for externally applying the active agent.

27. The system of claim 21, further comprising delivery interface means for delivering the active agent expelled from the third reservoir means to a biological interface.

28. The system of claim 27, further comprising a delivery control means for controlling delivery of the active agent by the delivery interface means according to rate, amount, or time of delivery of the active agent.
